(19) United States
(12) Patent Application Publication
(10) Pub. No.: US 2002/0026937 A1
(43) Pub. Date: Mar. 7, 2002

(54) Respiratory gas sensors in folw path

(76) Inventor: James R. Mault, Evergreen, CO (US)

Correspondence Address: Beverly M. Bunting, Esq., Gifford, Krass, Groh, Anderson & Citkowski, P.C., 280 N. Old Woodward, Suite 400, Birmingham, MI, 48009, US

(21) Appl. No.: 09/940,750

(22) Filed: Aug. 28, 2001

Related U.S. Application Data
(60) Provisional application No. 60/228,388, filed on Aug. 28, 2000.

(51) Int. Cl. A62B 7/00; A62B 9/00; A61M 15/00; A61M 16/00; A62B 18/00

(52) U.S. Cl. 128/200.24

(57) ABSTRACT

A respiratory gas meter is useful in detecting a gas component of a respiratory gas flowing along a flow path in the meter as a user breathes. The meter includes a respiratory gas sensor disposed in the flow path of the meter. One example of a respiratory gas sensor includes a fluorescence gas sensor having a radiation emitter for directing radiation along the flow path and a radiation detector for detecting fluorescence from the respiratory gas induced by the radiation. The respiratory gas sensor also includes a narrow band filter disposed between the detector and the gas, to pass fluorescence to the radiation detector, so as to instantaneously detect components of the respiratory gas passing through the flow path. Another example of a respiratory gas sensor includes a micromachined sensor that detect nitric oxide through the change in resonance frequency of the micromechanical structure due to absorption of nitric oxide on the surface of the structure.

### REFERENCE TO RELATED APPLICATION

This application claims priority of U.S. Provisional Application Serial No. 60/228,388 filed Aug. 28, 2000, entitled “Respiratory Gas Sensors in the Flow Path” and U.S. patent application Ser. No. 09/685,439 filed Oct. 11, 2000, entitled “Respiratory Nitric Oxide Meter” and is incorporated herein by reference.

### FIELD OF THE INVENTION

The present invention relates generally to the detection of gases in a flow path, and, in particular, to the detection of a gaseous component in a flow pathway of respiratory gas.

### BACKGROUND TO THE INVENTION

It is known that respiratory gas refers to the air that is inhaled and exhaled by an individual in a physiological process referred to as respiration. The composition of the inhaled air is known to be about 79% oxygen and 21% nitrogen. The composition of the exhaled breath of an individual includes oxygen and nitrogen, water vapor, carbon dioxide, and rare gases. In addition, minor quantities of nitric oxide (NO), nitrogen dioxide (NO₂), other nitrogen-containing compounds, sulfur-containing compounds, hydrogen peroxide, hydrogen, ammonia, ketones, aldehydes, esters, alkanes, and other volatile organic compounds may be present in exhaled breath. It should be appreciated that the term respired gas refers to either exhaled or inhaled gases.

Frequently, it is desirable to know the composition of the exhaled gas in order to diagnose and treat a predetermined medical condition in an individual, such as illnesses, inflammations (e.g. asthma), metabolic problems (e.g. diabetes), digestive processes, liver problems, kidney problems, gum disease, halitosis, blood component levels, and other or physiological conditions.

For example, it is frequently beneficial to measure a respiratory gas, such as the volume of oxygen in the exhaled breath. An example of an apparatus for measuring oxygen is a hand-held indirect calorimeter disclosed in commonly assigned disclosures, U.S. patent application Ser. Nos. 09/008,435, 09/601,589, and 09/630,389, which are incorporated herein by reference. The calorimeter includes an oxygen sensor to measure the oxygen content of the exhaled breath. One type of oxygen sensor known in the art is a fluorescence oxygen sensor. U.S. Pat. No. 3,612,866 to Stevens et al. describes oxygen concentration measurements based on oxygen quenching of molecular luminescence, which is incorporated herein by reference. The Gas Exchange Monitor (GEM) disclosed in Ser. No. 09/630,390 utilizes ultrasonic flow sensing and a fluorescence oxygen sensor to measure the oxygen consumption of a person, and hence their metabolic rate. The GEM also uses a novel flow path configuration, which is well suited for the detection of other respiratory gases.

Nitrogen and oxygen also form other compounds, especially during a physiological process. These typically take the form of Noₓ, where x represents an integer. Nitric oxide (NO) is a biochemically active molecule present in small quantities in exhaled air. Nitric oxide is beneficial in both the treatment and diagnosis of asthma and other forms of lung disorders. Asthma is a chronic disease characterized by intermittent, reversible, widespread constriction of the airways of the lungs in response to any of a variety of stimuli that do not affect the normal lung. A variety of drugs are commonly used to treat asthma. It is known that inhalation of nitric oxide (NO) is therapeutically beneficial in the prevention and treatment of asthma attacks and other forms of bronchoconstriction, of acute respiratory failure, or of reversible pulmonary vasoconstriction as discussed in U.S. Pat. No. 5,873,359 to Zapol et al., incorporated herein by reference. U.S. Pat. No. 5,904,938 and U.S. Pat. No. 6,063,407, both to Zapol et al. and incorporated herein by reference, disclose the use of inhaled nitric oxide in the treatment of vascular thrombosis and retinosis. Typically, treatment utilizing nitric oxide includes the introduction of nitric oxide as a portion of the respiratory gases being inhaled by the patient. The nitric oxide concentration is usually in the range of 1 to 180 parts per million (ppm). The difficulty presented in the administration of controlled amounts of nitric oxide is the determination of the concentration being introduced. It has traditionally been very difficult to quickly and accurately determine the concentration of nitric oxide in the gas mixture, especially where the concentration of nitric oxide is very low.

U.S. Pat. No. 5,839,433 to Higenbottam, incorporated herein by reference, describes the use of nitric oxide in the treatment of certain lung diseases and conditions. A drawback to the administration of gaseous nitric oxide is that it rapidly converts to nitrogen dioxide, a potentially harmful substance. Consequently, it is often preferable to intubate the patient so that nitric oxide is administered directly to the lungs. Whether or not intubated, it is very important to accurately monitor the amount of nitric oxide being introduced to the lungs. The Higenbottam '433 reference proposes an improvement wherein the nitric oxide is introduced as a short pulse of known volume, rather than continuously during inhalation.

U.S. Pat. No. 5,531,218 to Krebs, incorporated herein by reference, discusses the benefits of nitric oxide inhalation in the treatment of various disorders, including adult respiratory distress syndrome (ARDS). Krebs '218 discloses a system for administering nitric oxide that includes a source of nitric oxide, an analyzer for analyzing nitric oxide concentration, and a control unit, with the analyzer and the control unit cooperating to maintain the appropriate nitric oxide concentration. However, this system relies on the use of nitric oxide sensors utilizing infrared absorption measurement, electrochemical sensors, or chemiluminescence detectors. Each of these analyzers has drawbacks and cannot provide instantaneous nitric oxide concentration measurements.

Nitric oxide is also useful in the diagnosis of various physiological conditions. For example, the reversibility of chronic pulmonary vasorestriction may be diagnosed by administering known quantities of nitric oxide and monitoring changes in pulmonary arterial pressure (PAP) and cardiac output as described in U.S. Pat. No. 5,873,359 to Zapol et al.

Endogenous production of nitric oxide in the human airway has been shown to be increased in patients with asthma and other inflammatory lung diseases. Expired nitric oxide concentrations are also elevated in patients with reactive airways disease. Therefore, detection of nitric oxide is beneficial in diagnosing these conditions. However, proper diagnosis requires accurate measurement of nitric oxide in parts per billion (ppb) of gas-phase nitric oxide.

Determination of the level of nitric oxide is also beneficial in the diagnosis of inflammatory cnditions of the airways, such as allergic asthma and rhinitis, in respiratory tract infections in humans and Kartagener's syndrome. It also has been noted that the level of nitric oxide in the exhalation of smokers is decreased U.S. Pat. No. 5,922,610 to Alving et al., incorporated herein by reference, discusses the detection of nitric oxide in diagnosing these conditions, as well as gastric disturbances.

In addition to the above, nitric oxide may be used in the determination of lung function. For example, U.S. Pat. No. 5,447,165 to Gustafsson, incorporated herein by reference, explains that nitric oxide in exhalation air is indicative of lung condition. As one test of lung function, a subject may inhale a trace gas, such as nitric oxide. Then the concentration and time-dispersement of the gas in the exhalation air is measured. The shape of the curve representing the time dependent gas concentration in the exhalation air is indicative of lung function or condition. Obviously, it is necessary to have an accurate determination of both the concentration and the time-dependence of the concentration to allow for the most accurate diagnosis.

During exhalation, the gas mixture changes during the breath. The initial portion of the exhalation is “dead space” air that has not entered the lungs. This includes the respiritory gases in the mouth and respiratory passages above the lungs. Also, some portion of the exhalation measured by an analytical instrument may be attributed to dead air in the mask and flow passages of the apparatus. As a breath continues, respiratory gases from within the lungs are exhaled. The last portion or respiratory gases exhaled is considered alveolar air. Often it is beneficial to measure gas concentrations in alveolar air to determine various pulmonary parameters. For example, nitric oxide, as an indicator of various disease states, may be concentrated in the alveolar air. However, nitric oxide is also produced by various mucus membranes and therefore nitric oxide may be present in both the dead air space and in the alveolar air. During an exhalation, the dead air space may be overly contaminated with nitric oxide due to residence in the mouth and nasal cavities where nitric oxide is absorbed from the mucus membranes. Therefore, it is necessary to distinguish the various portions of exhalation for proper diagnosis. U.S. Pat. No. 6,038,913 to Gustafsson et al., incorporated herein by reference, discusses having an exhalation occur with very little resistance during an initial “dead space” phase of exhalation and then creating resistance against the remaining portion of the exhalation.

Numerous other approaches have been used or proposed for monitoring the concentration of nitric oxide in a gas mixture. These include mass spectroscopy, electrochemical analysis, colorimetric analysis, chemiluminescence analysis, and piezoelectric resonance techniques. Each of these approaches has shortcomings that make them poorly suited to widespread use in the diagnosis and treatment of disease.

In addition, a well known technique is detecting the chemiluminescence due to the reaction of NO with ozone (e.g. as used in U.S. patents to Gustafsson (U.S. Pat. No. 6,099, 480), Stamler et al. (U.S. Pat. No. 5,459,076) and Alving (U.S. Pat. No. 5,922,610)). Commercial devices are available from ECO Physics, Durnton, Switzwerland. However, ozone-reaction chemiluminescence sensors require a relatively complicated system, and ozone is a dangerous and toxic material. NO can also be detected by colorimetry. For example, U.S. Pat. No. 6,033,368 to Gaston IV et al., incorporated herein by reference, describes a condensate colorimetric nitrogen oxide analyzer. However, this device requires a cooled stage and is not well suited to time-dependent monitoring of a single breath. Metal oxide sensors for NO are known in the art. For example, U.S. Pat. No. 6,062,064 to Yoshida et al., incorporated herein by reference, describes the use on an SnO₂ sensor for measurement of low levels (ppb) of NO. However, the described system, including a catalyst, is not intended for respiratory analysis. Electrochemical cells are also known in the art, but may gave lower sensitivity. Commercial electrochemical devices are available from Innovative Instruments of Tampa, Fla. which use gas permeable membranes for selective NO response, but are only recommended for short-term measurement with gaseous samples.

Mass spectroscopy utilizes a mass spectrometer to identify particles present in a substance. The particles are ionized and beamed through an electromagnetic field. The manner in which the particles are deflected is indicative of their mass, and thus their identity. Mass spectorscopy is accurate but requires the use of very expensive and complicated equipment. Also, the analysis is relatively slow, making it unsuitable for real time analysis of exhalations. Preferably, in the breath-by-breath analysis of nitric oxide, it is desirable to quickly and accurately measure the nitric oxide concentration in the flow path as the gas mixture flows through the flow path. Mass spectroscopy requires sampling of portions of the gas mixture rather than analyzing the nitric oxide concentration in the flow pathway itself. Mass spectroscopy cannot be considered an instantaneous or continuous analysis approach. It requires dividing the exhalation into multiple discrete samples and individual analysis of each sample. This does not create a curve of the nitric oxide concentration but instead creates a few discreet points. Sampling-based systems are especially deficient when detecting gases in very low concentrations since large samples are required.

Electrochemical-based analysis systems use an electrochemical gaseous sensor in which gas from a sample diffuses into and through a semi-permeable barrier, such as membrane, then through an electrolyte solution, and then to one of typically three electrodes. At one of the three electrodes, a sensing redox reaction occurs. At the second, counter, electrode, a complimentary and opposite redox reaction occurs. A third electrode is typically provided as a reference electrode. Upon oxidation, or reduction, of the nitric oxide at the sensing electrode, a current flows between the sensing and counter electrode that is proportional to the amount of nitric oxide reacting at the sensing electrode surface. The reference electrode is used to maintain the sensing electrode at a fixed voltage. A typical electrochemical-based gas analyzer for detecting nitric oxide is shown is U.S. Pat. No. 5,565,075 to Davis et al., incorporated herein by reference. Electrochemical-based devices have high sensitivity and accuracy, but typically have a response time in excess of 30 seconds. This is significantly too slow to allow breath-by-breath, or continuous, analysis of respiration gases.

Colorimetric analysis relies on a chemical reaction by a gas which provides a corresponding change in pH, thereby triggering a color changed in an indicator. This approach requires expendable chemical substances. Also, this approach is often disturbed by the presence of other gases, particularly the relative amount of humidity present. Response times are too slow for analysis during a breath.

Chemiluminescent-based devices depend on the oxidation of nitric oxide by mixing the nitric oxide with ozone, O₃, to create nitrogen dioxide and oxygen. The nitrogen dioxide is in an excited state immediately following the reaction and releases photons as it decays back to a non-excited state. By sensing the amount of light emitted during this reaction, the concentration of nitric oxide maybe determined. An example of a chemiluminescent-based device is shown in U.S. Pat. No. 6,099,480 to Gustafsson, incorporated herein by reference. Chemiluminescent devices have response times as fast as about two hundred milliseconds, have high sensitivity, repeatability, and accuracy. However, similar to mass spectroscopy and electrochemical analysis, chemiluminescent analysis requires sampling of the gas mixture rather than continuous analysis of the gas concentration in the flow path itself. Also, chemiluminescent devices are typically very large and expensive.

Piezoelectric resonance techniques are sometimes referred to as MEMS (micro-electro-mechanical systems) sensor devices. Basically, a micro-etched cantilevered beam is coated with a “capture” molecule that is specific to the gas being analyzed. In theory, the capture molecule will capture the gas being analyzed in proportion to its ambient concentration. This alters the mass of the micro-etched cantilevered beam. Changes in mass of the beam may theoretically be detected based on changes in its resonant frequency. The change in resonant frequency should be directly proportional to the concentration of the gas being studied. A system for detecting air pollutants is disclosed in U.S. Pat. No. 4,111,036 to Frechette et al., incorporated herein by reference. While the theory behind piezoelectric resonance techniques is rather simple, there has been no known success to date in the analysis of nitric oxide concentrations.

U.S. Pat. No. 6,033,368 to Gaston IV et al. discloses an analyzer for measuring exhaled nitrogen oxides, nitrite and nitrate in very low concentrations. The analyzer includes a chilled exhalation passage which causes lung fluid vapors to collect. The resulting liquid is then analyzed using standard calorimetric assays. While somewhat simpler than other methods, the Gaston apparatus remains complicated, requiring pre-freezing of the chilling apparatus, and subsequent analysis of the collected liquid.

Commonly assigned U.S. Ser. No. 09/685,439, which is incorporated by reference, discloses a nitric oxide meter capable of continuously determining the nitric oxide concentration of a flow of respiratory gases in a flow pathway without the need for sampling the mixture. Advantageously, this meter provides nearly instantaneous response times so that analysis may be made during a breath or on a breath-by-breath basis. Thus there is a need in the art for a sensor for use in conjunction with a respiratory gas meter, such as a nitric oxide meter, for measuring the amount of respiratory gas present in a flow path.

### SUMMARY OF THE INVENTION

The present invention is a respiratory gas sensor for measuring a respiratory gas in a flow path of a respiratory gas meter. The meter includes a respiratory gas sensor disposed in the flow path of the meter. One example of a respiratory gas sensor includes a fluorescence gas sensor having a radiation emitter for directing radiation along the flow path and a radiation detector for detecting fluorescence from the respiratory gas induced by the radiation. The respiratory gas sensor also includes a narrow band filter disposed between the detector and the gas, to pass fluorescence to the radiation detector, so as to instantaneously detect components of the respiratory gas passing through the flow path. Another example of a respiratory gas sensor includes a micromachined sensor that detect nitric oxide through the change in resonance frequency of the micromechanical structure due to absorption of nitric oxide on the surface of the structure.

One advantage of the present invention is that a respiratory gas sensor is provided that measures the concentration of a respiratory gas present in the flow path of inhaled or exhaled air by an individual. Another advantage of the present invention is that the respiratory gas sensor is utilized in conjunction with a mechanism for analyzing and measuring respiratory gas concentration in a single breath. Still another advantage of the present invention is the respiratory gas sensor measures the concentration of nitric oxide present in the flow path during a single breath.

Other features and advantages of the present invention will be readily appreciated as the same become better understood after reading the subsequent description when considered in connection with the accompanying drawings.

### BRIEF DESCRIPTION OF THE DRAWINGS

FIG. 1 is a perspective view of a respiratory nitric oxide meter, for use with a respiratory gas sensor, according to the present invention;

FIG. 2 is a cross-sectional view of the meter of FIG. 1 taken along lines 2-2;

FIG. 3 is a perspective view of another embodiment of a nitric oxide meter for use with a respiratory gas sensor, according to the present invention;

FIG. 4 is a cross-sectional view of the meter of FIG. 3;

FIG. 5 is an exploded perspective view of an embodiment of a fluorescence-based nitric oxide sensor for use with a nitric oxide meter according to the present invention;

FIG. 6 is a cross-sectional side view of the sensor of FIG. 3 taken along lines 6-6;

FIG. 7 is an elevational view of still another embodiment of a respiratory gas sensor that utilizes laser detection, according to the present invention;

FIG. 8 is a schematic view of the sensor of FIG. 7 illustrating forwards and backwards fluorescence detection;

FIG. 9 is a schematic view of a filter for use with the sensor of FIG. 7;

FIG. 10 is a schematic view of still another embodiment of a respiratory gas sensor using photoionization, according to the present invention; and

FIG. 11 is a schematic view of a coaxial flow spirometer for use with a chemiluminescence respiratory gas sensor, according to the present invention.

### DETAILED DESCRIPTION OF THE PREFERRED EMBODIMENTS

In the present invention, a respiratory gas meter, such as the GEM or the nitric oxide meter, to be described, is combined with a respiratory gas sensor to improve the detection of respiratory components in an individual's breath. The respiratory nitric oxide meter provides for the measurement of the instantaneous nitric oxide concentration in a gaseous mixture as the mixture flows through a flow pathway. Unlike the prior art, the respiratory nitric oxide meter or GEM are not a sampling based analyzer, but instead measure the concentration of respiratory gas, such as nitric oxide or oxygen, in the flow pathway itself, and has a sufficiently fast response time so as to allow analysis on a breath-by-breath basis and to allow the monitoring of the changes in gas concentration during a single breath. Advantageously, the respiratory gas sensors used as part of the respiratory gas meter are considered instantaneous, with instantaneous being defined as fast enough to allow monitoring of changes in the nitric oxide concentration during a single breath. Investigation has indicated that response times of approximately 200 milliseconds (ms) or less are preferred in order to track changes in nitric oxide concentration, with 100 ms or less being even more preferred. Many of the prior art sensors and analyzers have response times on the order of several seconds, making them unsuitable for breath-by-breath analysis of the nitric oxide concentration of either inhalation of exhalation gases. Also, many are sampling based analyzers and therefore analyze discrete samples.

The present invention provides close correlation between gas concentration measurements and flow measurements, something not easily accomplished with prior art systems. In this example, the respiratory gas sensor is a nitric oxide sensor; however, the sensors described are applicable to other respiration components. Hence, the invention is not limited to NO sensing.

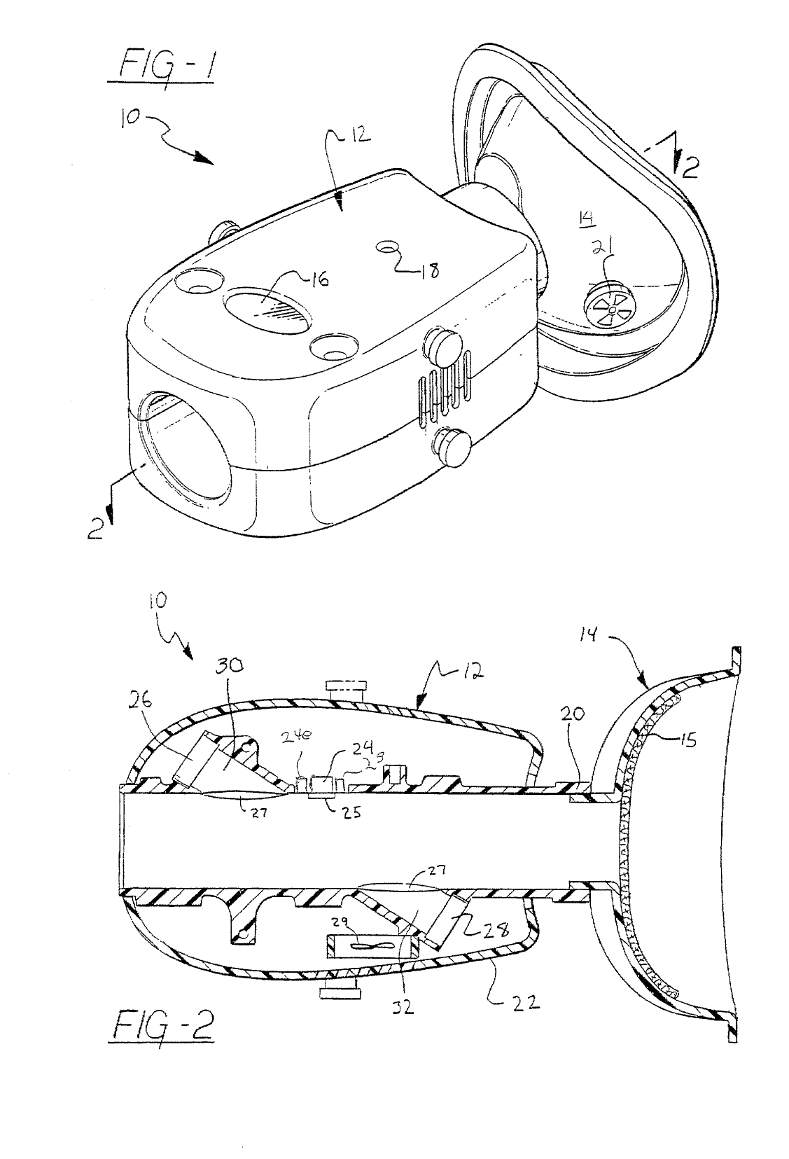

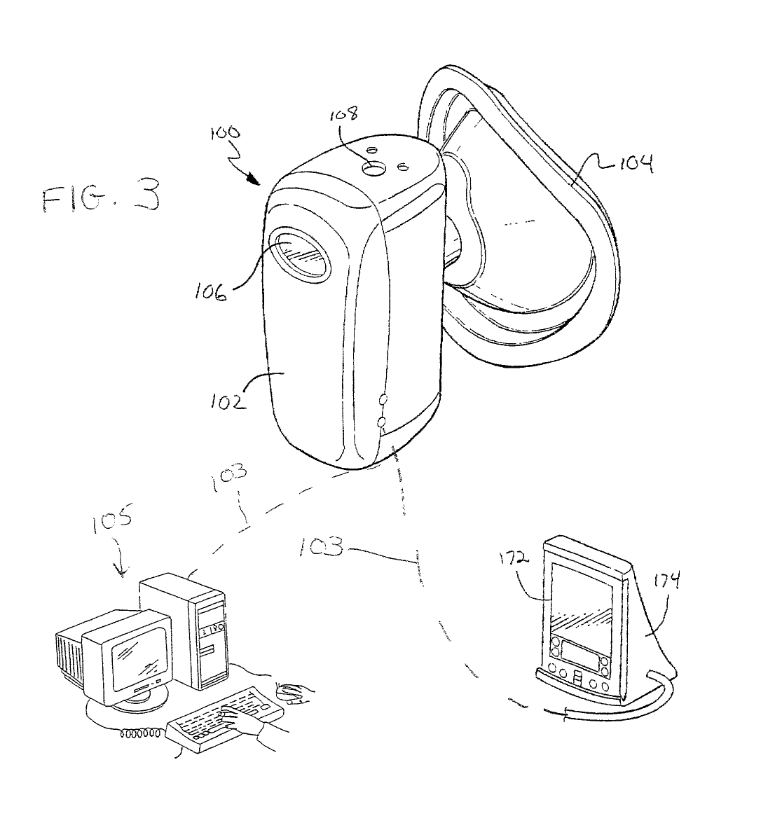

Referring to FIGS. 1 and 2, an example of a respiratory nitric oxide meter is generally shown at 10. The meter 10 includes a body 12 and a respiratory connector, such as a mask 14, extending from the body 12. Preferably, the meter 10 is a lightweight, handheld or wearable unit. In use, a user (not shown) grasps the body 12 and brings the mask 14 into contact with their face so that respiratory gases pass through the meter 10. Though not shown, straps may be provided for interconnecting the meter 10 with the user's face and head without the need to support it with a hand.

With the mask 14 in contact with the user's face, the user's inhalations and/or exhalations pass through the body 12 for analysis of the nitric oxide concentration. The meter 10 preferably includes a display 16 as well as a control button 18 for controlling operation of the meter 10.

Depending on the application, the meter 10 may be used to pass inhalation gases, exhalation gases, or both. In situations where it is preferred to pass only inhalation or exhalation gases, but not both, a valve 21 may be provided on the mask for allowing passage of the gases not to be analyzed. For example, the valve 21 may be a one-way valve that allows the passage of fresh air into the mask 14 upon inhalation but blocks exhalation, such that exhalation gases pass through the body 12 of the meter 10. By reversing the valve 21, exhalations may be passed through the valve while inhalations enter through the body 12. A second one-way valve may be provided in the body 12 for further directing gases. Without the valve 21, or with the valve 21 disabled, both inhalation and exhalation gases pass through the body 12.

Referring now to FIG. 2, the meter 10 is shown in cross-section so as to illustrate the internal construction. A flow pathway is formed through the body 12 by a generally straight flow tube 20. At one end, the flow tube 20 is interconnected with the mask 14, and its other end is open to the surrounding air. Alternatively, the second end of the flow tube 20 may be interconnected with a source and/or sink of respiratory gases, which may be referred to as a reservoir of respiratory gases. The term “reservoir” may also refer to the surrounding air. The body 12 includes an outer shell 22 which surrounds the majority of the flow tube 20 so as to provide an improved cosmetic appearance and to support a variety of additional components. As shown, the flow tube 20 is a generally cylindrical tube with a generally constant cross-section throughout its length. Consequently, inhalation and exhalation gases flow very freely into and out of the mask 14, thereby creating little resistance to natural respiration. A nitric oxide sensor 24, to be described, is disposed in the side of the flow tube 20, so as to be in contact with respiratory gases passing through the flow tube. The sensor 24 has a sensing face 25 positioned in a window or opening in the side of the tube.

In some embodiments of the present invention, a flow meter is also provided, so as to measure the flow of respiratory gases through the flow tube 20. Many types of flow meters may be used. However, in the preferred embodiment, an ultrasonic-based flow meter is used. Ultrasonic flow meters measure the instantaneous flow velocity of gas in a flow tube, thereby allowing determination of flow volumes. In the embodiment shown in FIG. 2, a pair of spaced-apart ultrasonic transducers 26 and 28 are disposed in the ends of a pair of side passages 30 and 32 which branch off of the flow tube 20. Ultrasonically transparent covers 27 may be provided where the side passages 26 and 28 intersect the flow tube 20 to reduce or prevent flow disturbances at the intersections. The ultrasonic transducers 26 and 28 and the side branches 30 and 32 are arranged such that ultrasonic pulses traveling between the transducers 26 and 28 pass through the flow tube 20 at an angle to its central axis. That is, ultrasonic pulses traveling between the transducers 26 and 28 travel along a path which is angled to the path of flow of respiratory gases through the flow tube 20. As shown, the side passages 30 and 32 essentially form an interrupted tube which intersects the flow tube 20 at an angle. As will be clear to those of skill in the art, ultrasonic pulses traveling between the transducers 26 and 28 have a component of their direction of travel which is parallel to the direction of flow of respiratory gases through the flow tube 20.

An example of how to measure flow velocity using ultrasonic pulses is described in U.S. Pat. Nos. 5,419,326; 5,503,151; 5,645,071; and 5,647,370, all to Harnoncourt et al., which are incorporated herein by reference. In the Harnoncourt patents, ultrasonic transducers are positioned so as to transmit pulses through a flowing fluid in a direction that has a component in the flow direction. Specifically, with fluid flowing through a tube, the transducers are positioned in the side walls of the tube at an angle such that ultrasonic pulses are transmitted at an angle to the fluid flow. Flow speed may be calculated based on the fact that ultrasonic pulses traveling with the flow travel faster while ultrasonic pulses traveling against the flow travel slower. Mathematical corrections are made for the fact that the ultrasonic pulses are traveling at an angle to the flow. Preferably, pulses are alternately transmitted in a direction with the flow and in a direction against the flow, so that a time difference may be calculated. The present invention may use ultrasonic transducers comprising a metalized polymer film and a perforated metal sheet. An example of such an ultrasonic flow measurement system is that supplied by NDD of Zurich, Switzerland and Chelmsford, Mass.

Ultrasonic pulses are transmitted with and against the direction of flow, resulting in measurement of upstream and downstream transit times. If the gas flow rate is zero, the transit times in either direction through the gas are the same, being related to the speed of sound and distance traveled. However, with gas flow present, the upstream transit times differ from the downstream transit times. For constant flow, the difference between sequential upstream and downstream transit times is directly related to the gas flow speed. Further details of this approach to ultrasonic flow sensing are described in a commonly assigned co-pending U.S. patent application Ser. No. 09/630,398, which is incorporated herein in its entirety by reference.

The meter 10 includes other components such as processing circuitry and the like that are disposed within the housing 12 for processing signals from the ultrasonic sensors 26 and 28. Also, a fan 29 may be provided to force fresh air over some of the internal circuitry. Preferably, the nitric oxide sensor 24 is positioned in the wall of the flow tube 20 approximately midway between the ultrasonic transducers 26 and 28. Therefore, the same portion of the flow is measured for flow speed and nitric oxide concentration at the same time, allowing coordination of the data.

It should be appreciated that the nitric oxide concentration sensor 24 may be located on the side of the flow tube 20 with no flow sensor. In this example, instantaneous nitric oxide concentrations are monitored during respiration, to provide a curve of nitric oxide concentrations. This data may be useful in the diagnosis and treatment of various diseases without obtaining flow data. The inclusion of flow sensors provide for determination of many additional parameters, including many respiratory parameters such as flow rate, flow volume, lung capacity, and others. For example, by including flow sensors, the meter can be used as a spirometer. The peak flow, the forced vital capacity (FVC), and the forced expiratory volume during the first second (FEV 1) may be derived from the collected data. The nitric oxide data, such as the time dependent concentration, may be combined with these parameters. Advantageously, concentration determinations for any respired component may be combined with flow readings from the ultrasonic transducers, or other flow meter, to calculate respired volumes.

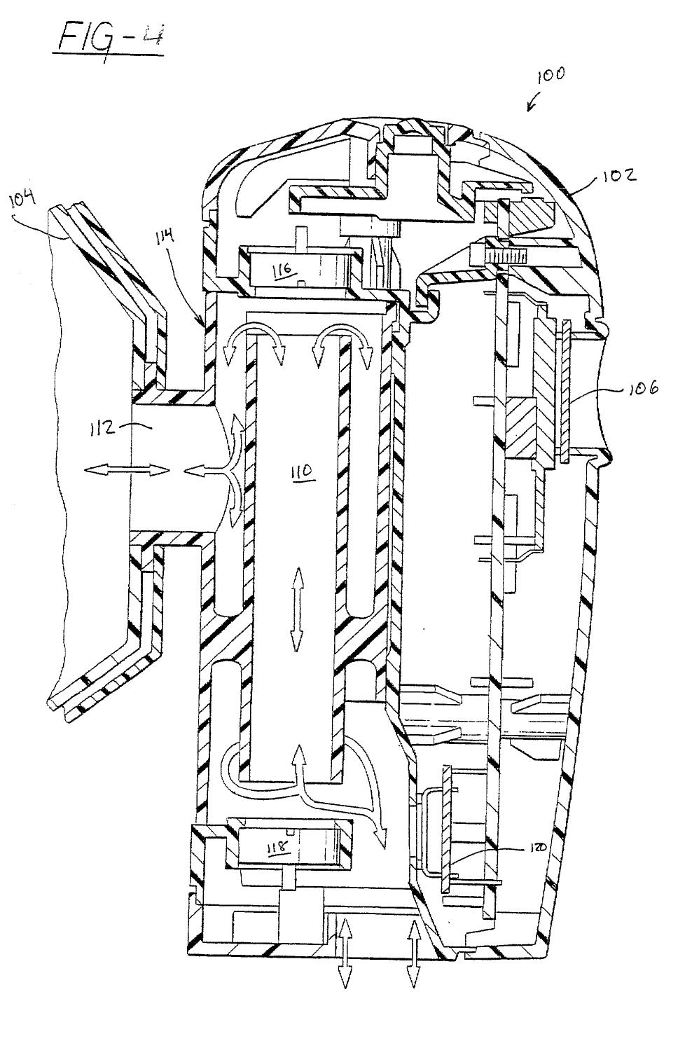

Referring to FIGS. 3 and 4, another example of a nitric oxide meter is generally shown at 100. This embodiment has a configuration similar to the configuration of the calorimeter described in Applicant's co-pending patent application Ser. No. 09/630,398, which is referred to as the GEM. The meter 100 includes a body 102 with a mask 104 extending therefrom. A display 106 is arranged on one side of the body 102 and a combination control button and indicator light 108 is disposed on another side of the body 102. It should be appreciated that calculation of flow velocity does not require correction for the flow sensors being arranged at an angle to the flow. In this example, the nitric oxide sensor 120 is positioned adjacent the flow pathway but below the bottom end of the flow tube 110. A nitric oxide meter according to the present invention may also be constructed in accordance with the other embodiments of the calorimeter discussed in Applicant's co-pending application Ser. No. 09/630,398, by substituting a nitric oxide sensor, as previously described, for the oxygen sensor used with a calorimeter. Other calorimeter designs that may be modified according to the present invention are disclosed in commonly assigned U.S. Pat. Nos. 4,917,108; 5,038,792; 5,178,155; 5,179,958; and 5,836,300, to Mault, and are incorporated herein by reference.

Preferably, data processing, storage, and analysis is performed by a remote computing device, such as a personal digital assistant (PDA) 172, as illustrated in FIG. 3. The PDA 172 is docked into an interface 174 which is connected to the meter 10, 100 by a communication link 103. Alternatively, data is transferred between the meter 10, 100 and the PDA 172 by wireless means or by transfer of memory modules, which store data, as described in Applicant's co-pending patent application Ser. No. 09/669,125, incorporated herein in its entirety by reference. Also, the respiratory gas meter 10, 100 may communicate with other remote computing devices 105, such as stationary or portable computers and remote devices such as servers via the Internet or dock or interconnect with a PDA, as also described in the co-pending application.

It is also contemplated that the nitric oxide meter 10 includes a graphic display 16 to show profiles of nitric oxide, breath flow, or other parameters for a period of time such as a single breath or one minute. Data may also be averaged over multiple breaths to provide an averaged profile. The meter 10, or other devices associated with the meter 10, may include a memory and a processor to store flow profiles or nitric oxide profiles indicative of various physiological conditions including a healthy normal state and various physiological disorders. The meter 10 or associated computational device may then compare the patient's data with the stored profiles in order to make a preliminary diagnosis. A PDA 172 may interconnect with the nitric oxide meter 10 and provide the necessary display and processing as well as diagnosis.

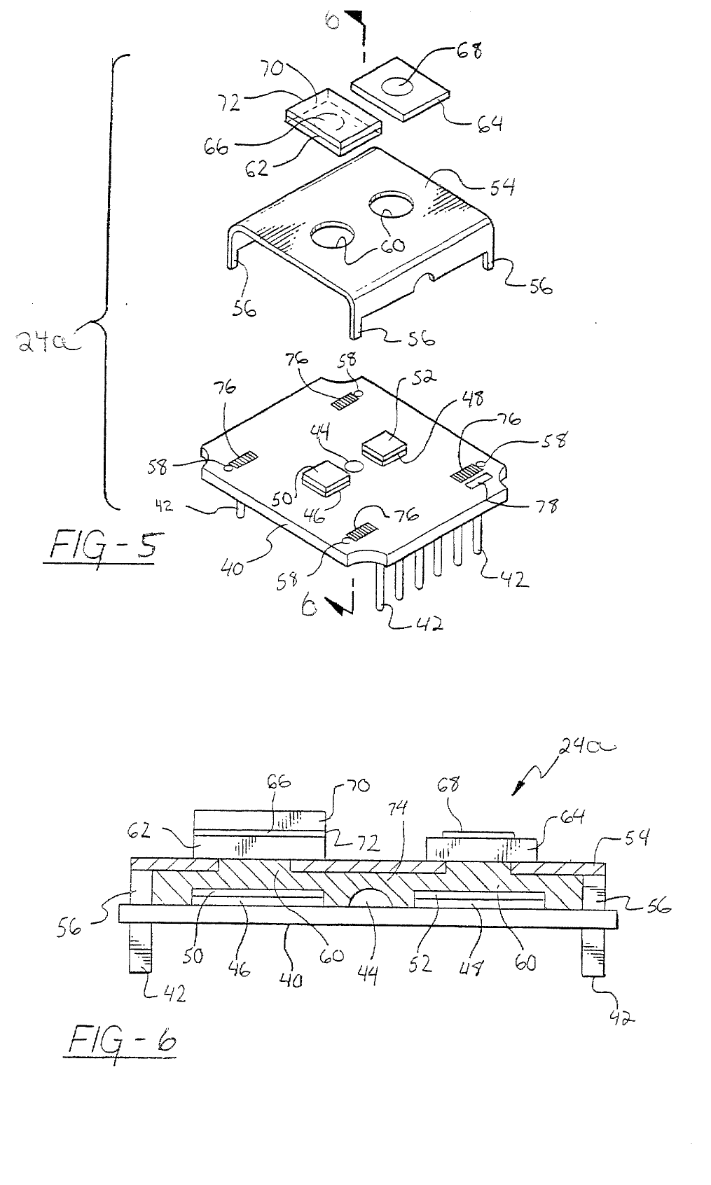

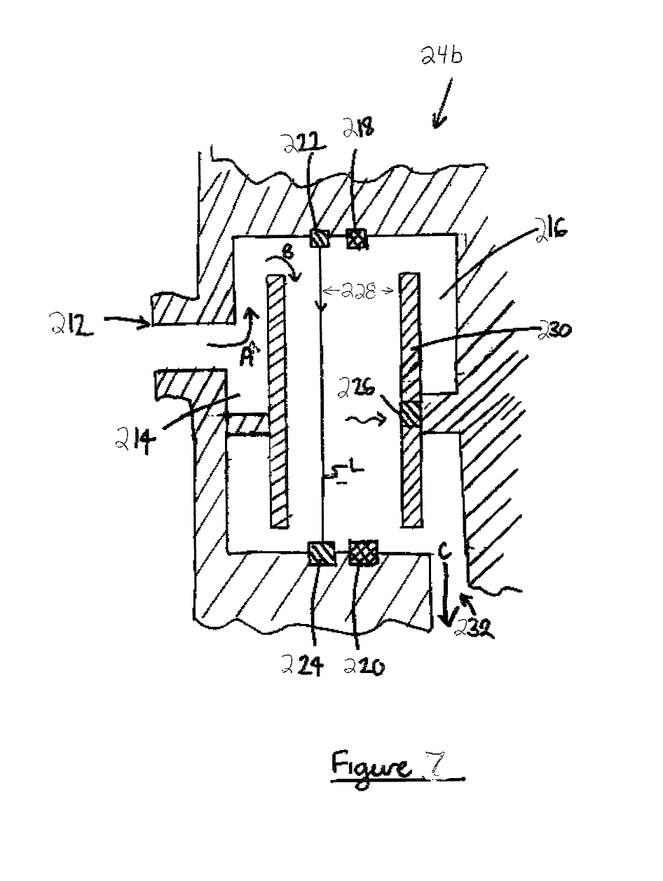

Referring now to FIGS. 5 and 6, one embodiment of a respiratory gas sensor 24, which is a fluorescence-based nitric oxide sensor 24a used to determine the partial pressure of nitric oxide in the respiration gases passing through the flow tube 20 is illustrated. Preferably, instantaneous nitric oxide concentration is measured at the same time flow is measured. Nitric oxide (NO) has an unpaired electron and interacts strongly with certain fluorescent compounds, for example transition metal complexes, to provide e.g. fluorescence wavelength changes, nonradiative de-excitation mechanisms (quenching), fluorescence lifetime changes, fluorescence onset time changes, etc. In some cases, interactions of NO with a fluorophore may result in emission at a new wavelength, which may then be detected. Changes in the peak emission wavelength may also be detected. One difficulty is selective sensitivity, i.e. it may be difficult to resolve fluorescence changes due to NO from changes induced by other atoms or molecules. Therefore, a nitric oxide permeable membrane (or combination of membranes) over the fluorescence quenching sensor is provided, which is impermeable to other gases which may cause fluorescence changes.

Fluorescence based oxygen sensors are known in the art, for example as described by Colvin (U.S. Pat. Nos. 5,517,313; 5,894,351; 5,910,661; and 5,917,605; and PCT International Publication WO 00/13003, all of which are incorporated herein by reference). A sensor typically comprises an oxygen permeable film in which oxygen-indicating fluorescent molecules are embedded. In U.S. Pat. Nos. 5,517,313 and 5,894,351, Colvin describes sensors using a silicone polymer film, and suggests using a ruthenium complex, tris(4,7-diphenyl-1,10-phenanthroline)ruthenium (II) perchlorate, as the oxygen indicator fluorophore molecule. The orange-red fluorescence of this ruthenium complex is quenched by the local presence of oxygen. Oxygen diffuses into the oxygen permeable film from the gas flowing over the film, inducing fluorescence quenching. The time response of the quenching effect, relative to concentration changes of oxygen in the gas outside the film, is related to the thickness of the film. Thin films are preferred for a rapid response, as described in U.S. Pat. No. 5,517,313.

The fluorescence-based nitric oxide sensor 24a has a chemistry adapted to detection of nitric oxide. A circuit board 40 has a plurality of pins 42 extending downwardly for interconnecting the sensor 24a with other components. An LED 44 is mounted generally to the center of the top of the circuit board. A pair of photodiodes 46 and 48 are also mounted to the top of the circuit board. The photodiodes are mounted symmetrically on opposite sides of, and a short distance from, the LED 44. An optical filter is mounted on top of each photodiode; filter 50 is mounted on photodiode 46 and filter 52 is mounted on photodiode 48. The optical filters preferably are bonded to the photodiodes with an optically clear adhesive.

A heat spreader 54, preferably a thin copper sheet with down-turned edges, is mounted to the top of the circuit board. The heat spreader 54 has a downwardly extending foot 56 at each of its four comers, each of which engage a hole 58 in the circuit board 40. The feet and the down-turned edges of the heat spreader 54 support the central portion of the heat spreader a short distance above the circuit board, leaving a gap therebetween. The LED 44, the photodiodes 46 and 48, and the filters 50 and 52 are disposed in this gap between the circuit board and the heat spreader. Two round holes 60 are cut in the heat spreader, one hole being directly above each of the photodiodes 46 and 48. Two pieces of glass substrate 62 and 64 are mounted to the top of the heat spreader 54, with one piece being mounted directly on top of each of the holes 60. As shown, these pieces of substrate 62 and 64 are square. A circle of fluorescent film is formed on top of each of the pieces of substrate; film circle 66 is formed on substrate 62 and film circle 68 is formed on substrate 64. A gas impermeable glass cover 70 is disposed over film circle 66 and bonded to the glass substrate 62 with epoxy 72. Therefore, film circle 66 is sealed in by the cover 70 above and the epoxy 72 at the edges. This results in one of the film circles, 68, being exposed to the surrounding atmosphere, while the other film circle, 66, is sealed in and not exposed. Therefore, film circle 66 does not react to changes in nitric oxide concentration while film circle 68 does. Film circle 68 will be referred to as a sensing region and film circle 66 will be referred to as a reference region. The substrates 62 and 64 and the materials applied to them form the sensing face of the sensor.

Referring again to FIG. 6, the gap between the circuit board 40 and the heat spreader 54, as well as the holes 60, are filled with an optically clear waveguide material 74. The waveguide material 74 serves to optically couple the LED 44 to the glass substrates 62 and 64, making the substrates an integral part of the waveguide. The waveguide material also optically couples the sensing region 68 and reference region 66 to the filters 50 and 52 and the photodiodes 46 and 48. The result is a continuous optical waveguide that optically couples these components. Suitable waveguide materials are manufactured by Norland Products of New Brunswick, N.J., and by Epoxy Technology of Bilerica, Mass., the latter under the name EPOTEK®.

In order to avoid condensation forming on the sensing region 68 and the reference region 66, the regions are preferably both warmed using the heat spreader 54. For this purpose, small heaters 76, comprising resistors, are mounted to the circuit board 40 adjacent each of the foot mounting holes 58. The heat spreader feet 56 are soldered into the holes and to the heaters 76, so that heat is transferred into the spreader 54. A thermistor 78 is mounted to the circuit board 40 in a position such that it contacts one of the down-turned edges of the heat spreader 54 when the sensor 24a is assembled. The thermistor 78 may be soldered to the edge to improve heat transfer. The thermistor 78 is then used to monitor the temperature of the heat spreader 54, and the heaters 76 are controlled so as to maintain a generally constant temperature. An EEPROM, containing calibration data for the sensor 24a, may be mounted to the underside of the circuit board 40.

The fluorescent films 66 and 68 are formed of materials whose fluorescence or absorbance characteristics change as a function of nitric oxide concentration. As an example, thiol or sulfhydryl may be joined to a fluorophore, such as pyrene, giving sulfhydrylpyrene. In this respect, an article entitled “Determination of Nitric Oxide Levels by Fluorescence Spectroscopy” by G. Gabor and N. Allon, published in the Biochemical, Pharmacological, and Clinical Aspects of Nitric Oxide (Edited by B. A. Weissman et al., Plenum Press, New York, 1995) is incorporated herein in its entirety.

Radiation from the LED is transmitted to the sensing region 68 and the reference region 66 by the optical waveguide material 74. The wavelength emission of the LED 44 is chosen to induce fluorescence from the fluorescent film regions 66 and 68. Fluorescence emissions from the sensing and reference regions, preferably shifted in wavelength compared to the LED radiation, are detected by the two photodiodes 46, 48. Photodiode 46 detects fluorescence from the reference region 66, and photodiode 48 detects fluorescence from the sensing region 68. The optical filters 50 and 52 overlie the photodiodes 46, 48, to pass the fluorescence radiation while rejecting other wavelengths, in particular the excitation radiation from the LED. The optical filters 50 and 52 may be an epoxy coating, a glass filter, or a polymeric-based sheet material. Preferably, a prefabricated polymeric-based sheet material is used. The emissions from the LED 44 and the fluorescence emissions from the films 66 and 68 pass through holes 60 in the plate 54. Preferably, the film circles 66 and 68, the holes 60, and the active areas of the photodiodes 46 and 48 are all circles of similar diameter.

During nitric oxide sensing measurements, the substrates 62 and 64 and sensing region 68 and reference region 66 preferably are maintained at a temperature sufficient to reduce problems associated with moisture condensation. The heating of the substrate is achieved by passing electrical current through the four surface-mounted resistors 76. The temperature of the copper plate 54 is monitored by the thermistor 78, allowing the heating current through the resistors and temperature to be regulated. If moisture was eliminated from the gas flow by some means, e.g. chemical drying, water absorbing/adsorbing substances, membranes, filters, foam sheets, etc., or prevented from condensing on the fluorescent film, such as by some surface treatment (a nitric oxide-permeable hydrophobic film or other approaches), then the sensor need not be heated.

The thin fluorescent films 66, 68 used in the nitric oxide sensor 24a respond very rapidly to changes in nitric oxide concentration thereby providing the sensor 24a with instantaneous response, as that term is defined herein. The sensor 24a has a response time preferably less than or equal to 200 milliseconds, and most preferably less than or equal to 100 ms. Even faster response times may be preferable for certain applications.

As will be clear to those of skill in the art, other types of nitric oxide concentration sensors may be used as long as they have an instantaneous response and are not sampling-based sensors. Also, the concentration of other component gases may be monitored using a meter similar to the one illustrated in the present invention. For example, an oxygen sensor may be added or may be substituted for the nitric oxide sensor so as to provide a calorimeter, as described in co-pending patent application Ser. No. 09/630,398.

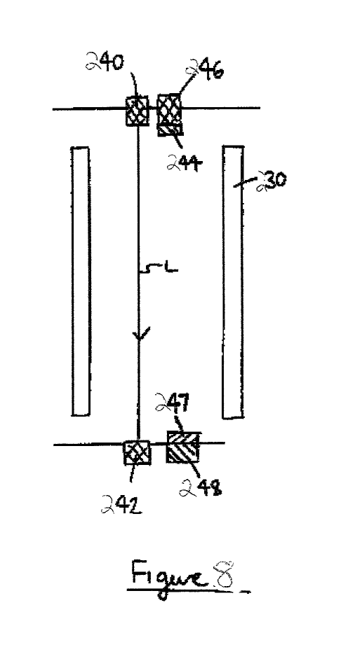

Another embodiment of a respiratory gas sensor 24 is a nitric oxide sensor 24b that utilizes laser detection in the coaxial flow path. Referring to FIG. 7, a laser source 222 is illustrated that produces a laser beam L propagating along the flow tube 20. The laser is absorbed by laser absorber 224. The laser source 222 may be used to excite atoms or molecular species in the flow tube 20. For example, the laser 230 includes an emission wavelength that induces photoexcitation of nitric oxide (NO). Fluorescence from the excited molecules is sensed by a detector 226. It should be appreciated that a filter (not shown) may be placed in front of the detector to pass fluorescence to the detector, while rejecting other wavelengths. Fluorescence or phosphorescence of excited molecules is detected, preferably in the IR or optical regions of the electromagnetic spectrum. It should also be appreciated that IR emissions from laser-excited molecules may be detected in some embodiments.

The laser source 222 is preferably a solid state laser, such as a semiconductor laser, but may also be a light-emitting diode or other electroluminescent source. The emission wavelength is preferably in the near-IR or visible regions of the spectrum, but may also be mid-IR, far-IR, UV, or elsewhere in the electromagnetic spectrum. Microwave radiation combined with magnetic fields in principle allows electron spin resonance detection of NO, though sensitivity will be low. The laser emission wavelengths are chosen for detection selectivity and sensitivity. The laser beam may undergo multiple reflections backwards and forwards through the flow tube 20 for increased levels of photoexcitation, fluorescence, or other factors increasing sensitivity, e.g. by replacing absorber 224 by a reflector. In addition, the laser emission may be modulated, with phase sensitive detection, for enhanced sensitivity. Should the laser emission be polarized, polarizers (not shown) are placed in front of the detector 226.

Advantageously, the sensor 246 is also suitable for Raman detection of molecules within the flow tube 20. In this case, a narrow band filter or dispersive element is placed in front of the detector 226 so that only Raman scattered light may reach the detector 226.

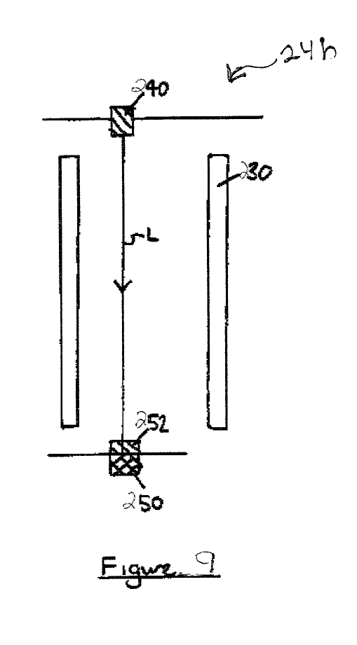

Referring to FIG. 8, the laser sensor 246 includes both backwards and forwards scattering/fluorescence detection. Laser radiation L from laser source 240 is absorbed by laser absorber 242. In back-scattering detection, a detector 246 adjacent to the laser 240 is used to detect fluorescence or scattered laser radiation. A filter 244 is used to transmit only radiation of interest to the detector 246. Preferably, polarized laser radiation and/or polarized detection is used, along with reflection of the laser beam, to increase path lengths through the flow tube 20. In the forward scattering/fluorescence geometry, the detector 248 and appropriate filter 247 are at the opposite end (to the laser) of the flow tube 20.

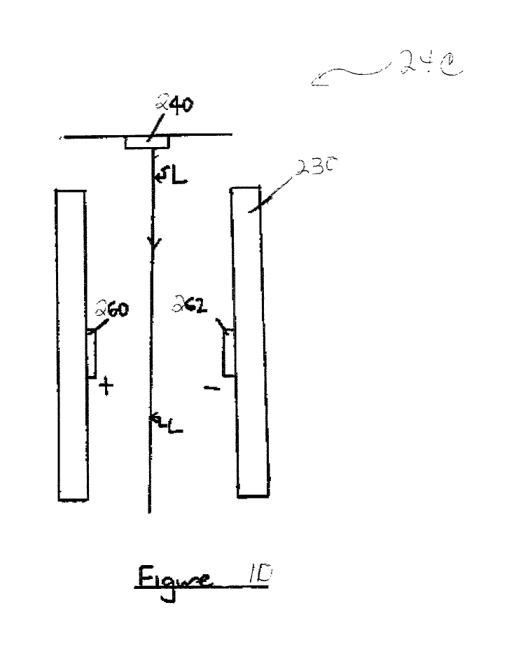

Referring to FIG. 9, a narrow band filter 252 is used to filter out (absorb or selectively reflect back) radiation from laser 240, allowing fluorescence or scattered radiation to pass through to the detector 250. Gas components in the flow tube 20 can also be detected using radiation absorption, e.g. if the filter 252 passes laser radiation to the detector 250. IR absorption is particularly useful for identifying carbon dioxide, ketones, and aldehydes using the fundamental or overtone absorption of the carbonyl group.

Still another embodiment of a respiratory gas sensor 24, such as a nitric oxide sensor 24c, utilizes a photoacoustic effect to detect nitric oxide NO. Pellaux et al. (U.S. Pat. No. 5,616,826) describe a system for photoacoustic detection of e.g. NO. Referring to FIG. 8, laser source 222 is modulated to produce laser pulses, at an appropriate wavelength depending on the gas component to be detected. At least one microphone is used to detect the photoacoustic signal. It is contemplated that the microphone could be incorporated in the sensor using micromachining technology, such as micromachined ultrasonic transducers as shown at 218 and 220.

Advantageously, micromachined ultrasonic transducers 218, 220 may be used to investigate the ultrasonic spectra of respired gases over broad frequency ranges, e.g. 50 kHz-10 MHz. Resonances due to individual molecular species may be detected and used to determine the concentration of that species in the respired gases.

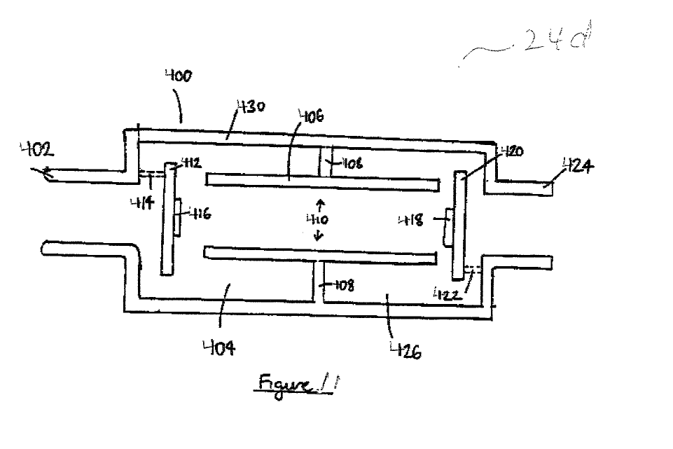

Referring to FIG. 10, yet another embodiment of a respiratory gas sensor 24, and in particular a nitric oxide sensor 24c, in which selective photoionization of a chosen molecular or atomic species is used for detection of the respiratory gas, such as nitric oxide. The emission wavelength of laser 240 is chosen so that only the required analyte is photoionized. The laser beam L passes along the flow column formed by flow tube 230. A high voltage is applied between two electrodes 260 and 262, mounted on the flow tube 230. The current detected flowing across from one electrode to the other is proportional to the concentration of ionized species. Electric discharges within the flow path may also be used for selective ionization of molecules or radicals (e.g. NO).

It is contemplated that if the species to be detected is known, a system configuration of e.g. laser photoionization, electric and/or magnetic fields, and ion detecting apparatus can be designed specifically to detect an analyte of known mass/change ratio. Such a simplified configuration would be considerably less expensive than a conventional instrument, and suitable for respiratory analysis of e.g. NO. Advantageously, conventional mass spectrometers can also be used in conjunction with a respiratory meter, such as the GEM, e.g. by connecting to the source/sink of respired gases.

A further embodiment of a respiratory gas sensor 24, such as a nitric oxide sensor 24d, utilizes chemiluminescence detection. It is known that nitric oxide is detectable using the chemiluminescence produced by the reaction with ozone. Unlike the other embodiments described here, this technique is fairly specific to NO detection.

For example, the GEM 100 can be combined with a commercial nitric oxide detector for detection of NO. A commercial chemiluminescence NO detector can be attached to the source/sink of respiratory gases. A spirometer version of the GEM, useful for the accurate measurement of breathing volume, may also be combined with a NO detector.

FIG. 11 shows an in-line coaxial flow spirometer 400 which, for example, may be combined with a commercial NO detecting instrument. Exhaled gas enters through mouthpiece 402, and enters concentric chamber 404. Concentric chambers 404 and 426 are separated by annular divider 408. For unidirectional gas flow, only the concentric chamber 404 is preferred, however, for bidirectional flow, chamber 426 is also preferred. Gas flows into the flow column 410 formed largely by flow tube 406. Gas flow volume and direction is determined using ultrasonic transducers 416 and 418, as is known in the art (e.g. U.S. patents to Harnoncourt, such as U.S. Pat. No. 5,645,071, and described in applications to J. R. Mault et al.). Transducer 416 is supported by flow column end piece 412, which is itself supported by a piece or pieces such as 414, which do not significantly block the air flow, but which provide mechanical association between the flow column end piece 412 and the spirometer housing 430, and which also allow electrical contact to the transducer 416. Piece 414 may be a spoke, rib, column, etc. Likewise, transducer 418 is supported by flow column end piece 420, which is supported by a piece or pieces such as 422 which do not significantly block the air flow. Exhaled gases exit the spirometer through exit vent 424. The gases exiting through vent 424 may then be passed to another analytical instrument, such as a mass spectrometer, or commercial NO detector.

Alternatively, a commercial electrochemical probe may be placed in the flow path 410 via a port in the side of the coaxial spirometer. Electric discharges within the flow path may be used for ozone production, which forms detectable chemiluminescence in its reaction with NO.

Ozone may also be introduced into the flow column, and the chemiluminescence detected. For example, ozone may be passed through a tube through the flow path (or into a chamber inside the flow path), the material of which allows ozone to diffuse out, or (preferably) NO to diffuse in, whereby the resulting chemiluminescence may be detected. The pressure inside the tubing or chamber may be below atmospheric. Tubing material, or a reflective material on the inside surface, may be used as a light guide to convey the chemiluminescence radiation to a detector. Alternatively, ozone may be generated within the flow path by a conventional method (e.g. using an ozonizer), and chemiluminescence detected.

Still a further embodiment of a respiratory gas sensor 24, such as a nitric oxide sensor, is a micromachined sensor 24e, as shown in FIG. 2. In U.S. Pat. Nos. 6,050,722; 6,016,686; 5,918,263; 5,719,324; 5,445,008, and related applications, Thundat and co-inventors describe micro-mechanical sensors which may be adapted for NO or other respiration component detection, e.g. through the change of resonance frequency of a micromechanical structure due to gas adsorption on the surface (alternatively absorption, chemisorption, physisorption, etc., on or in the surface). Micromechanical sensors 24e may also be used in temperature sensing (e.g. U.S. Pat. No. 6,050,722). Preferably, the micromachined sensor 24e is placed along the flow tube 20 to detect trace respiration components. It is contemplated that the micromachined sensor 24e may also be fabricated containing some combination of ultrasonic transducers, pressure sensors, humidity sensors, trace gas sensors, and temperature sensors, which are useful in respiration analysis.

It is known that there may be small quantities of glucose in exhaled breath. These quantities are related to blood glucose levels, due to processes e.g. within the lungs. Hence, a glucose sensor 23 may be disposed in the flow path. Examples of glucose sensors 23 include a fluorescence sensor, colorimetric sensor, micromechanical sensor, or other sensor technology e.g. using enzymes such as glucose oxidase. For example, a micromachined sensor may have a surface coated with glucose-binding chemistry.

Advantageously, the respiratory meter 10 with respiratory gas sensor 24 is useful in diagnosing and monitoring respiratory disease such as asthma. Volume, flow, and NO content of respired gases are useful diagnostic indicators, particularly when combined. For example, the volume and flow rate of inhaled or exhaled breath may be monitored using a respiratory spirometer, preferably using the coaxial flow design of the GEM 100, but not necessarily including an oxygen or carbon dioxide sensor. An NO sensor is preferably combined with the spirometer. The flow rate is monitored as a function of time, and the data transferred to the PDA 172, e.g. using a wireless transfer (e.g. Bluetooth), IR, cables, or transfer of a memory medium. The flow rate for a single breath, or a number of averaged breaths, may then be plotted and analyzed on the PDA 172. Alternatively, the GEM 100 may be provided with a display for respiratory flow/volume graphing vs. time, and with data analysis functionality (such as pre-loaded software) for determining parameters such as peak flow from the collected data.

Nitric oxide content of exhaled breath may also be monitored as a function of time, and in a similar way displayed on a PDA 172, or on the GEM 100. The peak flow rate, the forced vital capacity (FVC), and the forced expiratory volume in one (the first) second (FEVI) may be derived from the collected data. Nitric oxide data, e.g. the time-dependent concentration over a breath, may be combined with these parameters. The data may be transferred from the GEM 100, PDA 172, or a combined device comprising the functionality of both, to a remote computer system using a communications network connection such as a wireless Internet connection. A physician or other health professional may view the collected data, e.g. using an Internet connection, and provide advice to the person e.g. in terms of self-administration of medication. For a child, data may be sent to a pediatrician. This scheme is extremely useful for diagnosing childhood asthma. Chronic obstructive pulmonary diseases in adults may also be usefully diagnosed.

Administration of NO is sometimes useful in treating inflammatory diseases. Hence, a spirometer with NO measuring capability may be useful in both diagnosing and treating the condition, the latter through determination of the volume of NO administered.

Advantageously, the respiratory gas meter 10 with respiratory gas sensor 24 is useful for various types of medical monitoring. For example, the respiratory gas meter may be combined with a ventilator for measuring the volume and composition of gas supplied to a person. NO is also produced in the intestines, and levels are enhanced by inflammation of the colon and rectum, as discussed by Alving et al. (U.S. Pat. No. 6,063,027). Hence, it may be useful to monitor NO levels in intestinal gas, e.g. flatulence. Another example is the use of radio luminescent sensors to perform xenon lung function tests using the flow geometry of the GEM 100. Still another example is the use of the respiratory gas sensor to detect NO in exhaled breath, to calibrate or zero the meter using the inhaled breath. Atmospheric air normally contains negligible amounts of NO, so this can be used to ensure a zero reading from the detector for inhalation readings. In addition, calibrated dilutions of NO may also be passed through the meter for detector calibration.

The present invention has been described in an illustrative manner. It is to be understood that the terminology, which has been used, is intended to be in the nature of words of description rather than of limitation.

Many modifications and variations of the present invention are possible in light of the above teachings. Therefore, within the scope of the appended claims, the present invention may be practiced other than as specifically described.

1. A respiratory gas meter for detecting a gas component of a respiratory gas flowing in a flow path of the meter as a user breathes, with a respiratory gas sensor disposed in the flow path, said respiratory gas sensor comprising:
a fluorescence gas sensor having a radiation emitter for directing radiation along the flow path and a radiation detector for detecting fluorescence from the respiratory gas induced by the radiation; and
a narrow band filter disposed between the detector and the gas, to pass fluorescence to the radiation detector, to instantaneously detect components of the respiratory gas passing through the flow path.

2. A respiratory gas meter as set forth in claim 1, wherein said fluorescence gas sensor further comprises:
a fluorescent material that changes in fluorescence in response to changes in the level of nitric oxide;
a radiation source means that induces fluorescence in the fluorescent material;
a nitric oxide permeable membrane disposed between the fluorescent material and the gas flow, so that nitric oxide from the gas flow interacts with the fluorescent material;
a detector means for detecting fluorescence from the fluorescent material; and
signal processing circuitry for detecting changes in the fluorescence due to the presence of nitric oxide in the respiratory gas of the user.

3. The respiratory gas meter as set forth in claim 2, wherein the fluorescent material is a transition metal complex.

4. The respiratory gas meter as set forth in claim 1 wherein a signal representing the sensed respiratory gas component is transmitted to a remote computing device.

5. The respiratory gas meter as set forth in claim 4 wherein said remote computing device is a personal digital assistant.

6. A respiratory gas meter for detecting a gas component of a respiratory gas flowing in a flow path of the meter as a user breathes, with a respiratory gas sensor disposed in the flow path, said respiratory gas sensor comprising:
a fluorescence quenching gas sensing means having a radiation emitter for directing radiation along the flow path and a radiation detector for detecting fluorescence from the respiratory nitric oxide gas induced by the radiation;
a fluorescent material that changes in fluorescence in response to changes in the level of nitric oxide;
a radiation source means that induces fluorescence in the fluorescent material;
a nitric oxide permeable membrane disposed between the fluorescent material and the gas flow, so that nitric oxide from the gas flow interacts with the fluorescent material;
a detector means for detecting fluorescence from the fluorescent material; and
signal processing circuitry for detecting changes in the fluorescence due to the presence of nitric oxide in the respiratory gas of the user.

7. The respiratory gas meter as set forth in claim 6, wherein the fluorescent material is a transition metal complex.

8. The respiratory gas meter as set forth in claim 6 wherein a signal representing the sensed respiratory gas component is transmitted to a remote computing device.

9. The respiratory gas meter as set forth in claim 8 wherein said remote computing device is a personal digital assistant.

10. A respiratory gas meter for detecting a gas component of a respiratory gas flowing in a flow path of the meter as a user breathes, with a respiratory gas sensor disposed in the flow path, said respiratory gas sensor comprising:
a micromachined gas sensor disposed in the flow path to instantaneously detect components of the respiratory gas passing through the flow path by a change in resonance frequency of a micromechanical structure due to gas absorption on the surface of the structure.

11. A respiratory gas meter as set forth in claim 10, wherein said micromachined sensor detects nitric oxide in the respiratory gas.

12. The respiratory gas meter as set forth in claim 11 wherein a signal representing the sensed respiratory gas component is transmitted to a remote computing device.

13. The respiratory gas meter as set forth in claim 12 wherein said remote computing device is a personal digital assistant.
